(19) United States
(12) Patent Application Publication
(10) Pub. No.: US 2026/0367375 A1
(43) Pub. Date: Nov. 19, 2026

(54) SURGICAL MICROSCOPE, METHOD FOR OPERATING A SURGICAL MICROSCOPE

(71) Applicant: CARL ZEISS MEDITEC AG, Jena (DE)

(72) Inventor: Remzi OEZDEMIR, Oberkochen (DE)

(21) Appl. No.: 19/676,577

(22) Filed: May 13, 2026

(30) Foreign Application Priority Data
May 13, 2025 (DE) 102025118314.7

(51) Int. Cl. G02B 21/00; G02B 21/36

(52) U.S. Cl. CPC G02B 21/0012; G02B 21/361; G02B 21/368

(57) ABSTRACT

A surgical microscope has a microscope body and an attachment. The attachment is detachably attached to the microscope body by way of an interface. The microscope body is provided with an optical arrangement and an image sensor. The optical arrangement guides an observation beam path that extends between an object plane and the image sensor. The attachment comprises a display for displaying image data acquired by the image sensor. The interface comprises a separable electrical channel and a separable optical data channel. The image data are transmitted along the optical data channel via the interface. The present disclosure also relates to a method for operating a surgical microscope.

### CROSS-REFERENCE OF THE DISCLOSURE

The present patent application claims the priority of the German patent application DE 10 2025 118 314.7 filed on 13 May. 2025, with reference being made thereto and the content thereof being incorporated in full here ("incorporation by reference").

### FIELD OF THE DISCLOSURE

The present disclosure relates to a surgical microscope and a method for operating a surgical microscope.

### BACKGROUND OF THE DISCLOSURE

Surgical microscopes are used to provide a user with a magnified image representation of a body region of a patient to be treated. These days, an uninterrupted optical observation beam path that extends from the object plane of the surgical microscope to the eyepieces into which the user gazes are no longer mandatory. Instead, the microscope image can be transmitted by way of a transfer of image data in a portion of the route. The conditions in such a microscope, in which the observation beam path extends as far as an image sensor and the image data acquired by the image sensor are displayed on a display, differ from the conditions in a conventional surgical microscope, as described for example in EP 1 892 553 A1, and differ even more from the conditions in conventional microscopes serving other uses, for example as described in JP 2001 215 416 A.

In order to be able to use the surgical microscope flexibly, the display on which the image captured by the image sensor is displayed can be a constituent part of an attachment which is detachably connected to the microscope body of a main component of the surgical microscope. For example, the attachment can be a tube on which the eyepieces for a user of the surgical microscope are formed. The tube can be changed with little effort if said tube is designed as an attachment such that, for example, it is possible to use a tube selected appropriately for the respective application or the respective user.

It has not proved entirely straightforward to design the interface between such an attachment and the microscope body in such a way that a transmission of image data between the image sensor and the display is made possible.

The problem addressed by the present disclosure is that of presenting a surgical microscope and a method for operating a surgical microscope which reduce the aforementioned deficiencies. The problem is solved by the features of the independent claims. Advantageous embodiments are specified in the dependent claims.

### SUMMARY OF THE DISCLOSURE

A surgical microscope according to the present disclosure comprises a microscope body and an attachment, wherein the attachment is detachably attached to the microscope body by way of an interface. The microscope body is provided with an optical arrangement and with an image sensor. The optical arrangement guides an observation beam path that extends between an object plane and the image sensor. The attachment comprises a display for displaying image data acquired by the image sensor. The interface comprises a separable electrical channel and a separable optical data channel. The image data are transmitted along the optical data channel via the interface.

The present disclosure proposes the use of an optical data channel for the transmission of the image data between the microscope body and the attachment. The optical data channel can be used to transmit modulated light signals whose information content corresponds to the image data. The light signals can be processed in such a way in the attachment that a representation of the image data on the display is rendered possible. The interface additionally comprises an electrical channel via which, for example, the electrical energy required for operation of the display can be transmitted. Both the electrical channel and the optical data channel are separated from one another at the interface when the attachment is detached from the microscope body. The attachment can be quickly and easily replaced with another attachment.

The image sensor can be designed to provide the image data, which correspond to the microscope image, in the form of an electrical signal. The surgical microscope can comprise a first transducer, by means of which the electrical signal of the image data is converted into an optical signal of the image data. The surgical microscope can comprise an optical data channel which extends from the first transducer into the attachment via the interface. The image data transmitted via the optical data channel can be supplied to the display such that a reproduction of the microscope image can be displayed on the display. A second transducer used to convert the light signal transmitted via the optical data channel into an electrical signal can be arranged between the optical data channel and the display. The image data can be supplied to the display in the form of an electrical signal.

A separation point corresponding to the interface can be formed in the optical data channel. A first section of the optical data channel and a second section of the optical data channel may adjoin the separation point without being directly physically connected to one another. The first section of the optical data channel and the second section of the optical data channel can abut one another at the separation point such that the image data are transmitted via a common interface between the first section of the optical data channel and the second section of the optical data channel. It is also possible for the first section and the second section of the optical data channel to be at a distance from one another such that the light signal is transmitted over an air gap between the first section and the second section.

The optical data channel can comprise a block made of a material transparent to the light signal. For example, the block can be a glass block. The block can be a constituent part of the microscope body or a constituent part of the attachment. The surgical microscope can comprise a first transparent block and a second transparent block, wherein the first transparent block is a constituent part of the microscope body, and the second transparent block is a constituent part of the attachment. A light signal output by the first transducer can travel to the second transducer through the first block and the second block. The first transducer and the second transducer can be arranged adjacent to the first block and the second block such that the optical data channel does not comprise any further optical elements other than the two blocks. The term block expresses the fact that an optically shaped surface need not necessarily be present, as would be the case with a lens element, for example.

In addition to that, or as an alternative, the optical data channel can comprise one or more light guides. A first light guide can extend between the first transducer and the interface. A second light guide can extend between the interface and the second transducer. One or more of the light guides may consist of a polymer material should the transmission path for the light signal be sufficiently short such that said light signal is not excessively attenuated in the polymer material. Optical waveguides made of glass fibre, which allow longer transmission paths, are also possible. In particular, a light guide made of glass fibre can be used if the first transducer is not arranged within the microscope body but rather at a position spaced apart therefrom, for instance in a stand of the surgical microscope. Transmitting the image data by light signals rather than electrical signals has advantages in relation to electromagnetic compatibility.

The electrical channel can be designed to transmit the electrical energy required for the operation of the display. The electrical channel may comprise a first pin and a second pin, which are part of a common circuit. The electrical channel may comprise a first section extending within the microscope body and a second section extending within the attachment. The second section of the electrical channel may be connected to the display. The first section of the electrical channel may be connected to an electric current source. The transmission of the electrical energy between the current source and the first section of the electrical channel can be effected by way of a control unit of the surgical microscope and/or be controlled by a control unit of the surgical microscope.

The electrical channel can also be used to transmit the electrical energy required for operation of the second transducer. The second transducer can be connected to the electrical channel in a similar manner to the display. In one embodiment, the electrical channel comprises a plurality of electrical conductor paths, with some of the electrical conductor paths being used to supply the display and other electrical conductor paths being used to supply the second transducer.

In addition to that, or as an alternative, the interface can be used to transmit control information between the attachment and the microscope body. For example, such control information can be used to activate or deactivate the display, to activate or deactivate the transducer and/or to transmit information about the state of elements of the surgical microscope. For example, information about the state of the display, of the first transducer, of the second transducer, of the optical arrangement of the surgical microscope, of an illumination system of the surgical microscope and/or of further components of the surgical microscope can be of interest.

In one embodiment, the interface is used to transmit control information in the form of an identification code which allows identification of the attachment connected to the microscope body. The information can be processed in the control unit of the surgical microscope in order to control the operation of the surgical microscope in a manner suitable for the identified attachment.

The control information can be transmitted via the electrical channel of the interface. This is advantageous because the electrical channel can be operated with less outlay than the optical data channel. It is thus possible to transmit information via the electrical channel without the entire operating system of the surgical microscope having to be active. Technically, it is also possible to transmit control information via the optical data channel. In order to avoid the associated increased outlay, the optical data channel is used exclusively for the transmission of the image data in one embodiment, and so no other information is transmitted via the optical data channel.

The interface can comprise a mechanical connector used to hold the attachment on the microscope body. The mechanical connector can comprise a positioning structure and a locking mechanism. The positioning structure and the locking mechanism can be matched to one another such that the locking mechanism can only be locked when the positioning structure is engaged. When the attachment is attached to the microscope body, the attachment can initially be positioned relative to the microscope body such that the positioning structure is in engagement. The locking mechanism can be locked in a subsequent step. Conversely, when the attachment is detached from the microscope body, locking mechanism detachment can be implemented first, followed by the attachment being moved relative to the microscope body such that the positioning structure is also freed.

The positioning structure may comprise a projection and a holding element, behind which the projection engages. The projection can be formed on the attachment, and the holding element can be formed on the microscope body. The reverse design is likewise possible.

The positioning structure can have a first engagement position and a second engagement position, wherein it is not yet possible to lock the locking mechanism in the first engagement position, whereas the locking mechanism can be locked in the second engagement position. The transition between the first engagement position and the second engagement position can be implemented by way of a pivoting movement of the attachment relative to the microscope body. The pivoting movement can be implemented about a pivot axis which forms an angle with a central axis of the interface, in particular a right angle.

Within the interface, the distance between the optical data channel and the pivot axis can be greater than the distance between the central axis of the interface and the pivot axis. The distance between the electrical channel and the pivot axis can be greater than the distance between the central axis of the interface and the pivot axis. This design contributes to the electrical channel or the optical data channel being connected at the separation point by way of a defined movement, as a result of which the risk of incorrect operation and damage is reduced. The interface of the electrical data channel can be designed such that an earth contact is brought into engagement, and so electrostatic charges on the attachment can be dissipated before a circuit is closed via the electrical channel.

The interface between the attachment and the microscope body can be provided with a continuous peripheral seal. The continuous peripheral seal may be designed to prevent the ingress of liquids into the interior of the interface. The optical data channel and the electrical channel can be arranged within the continuous peripheral seal.

The present disclosure also relates to a method for operating a surgical microscope, wherein the surgical microscope comprises a microscope body and an attachment and wherein the attachment is detachably attached to the microscope body by way of an interface. Image data representing images from an object plane of the surgical microscope are acquired by an image sensor. The image data are transmitted across the interface to a display formed in the attachment. The image data are transmitted via an optical data channel formed in the interface. The display is supplied with electrical energy via an electrical channel formed in the interface.

The disclosure encompasses developments of the method with features that are described in the context of the surgical microscope according to the present disclosure. The disclosure encompasses developments of the surgical microscope with features that are described in the context of the method according to the present disclosure.

### BRIEF DESCRIPTION OF THE DRAWINGS

The disclosure, and its advantages and drawings, will be better understood from the following description of representative embodiments together with reference to the accompanying drawings. These drawings depict only representative embodiments and are therefore not to be considered as limitations on the scope of the various embodiments or claims.

FIG. 1 shows a schematic view of a surgical microscope, according to aspects of the present disclosure.

FIG. 2 shows aspects of the surgical microscope from FIG. 1 in a schematic illustration, according to aspects of the present disclosure.

FIG. 3 shows the view in accordance with FIG. 2 in a different state of the surgical microscope, according to aspects of the present disclosure.

FIGS. 4-6 show a schematic illustration of the sequence when attaching an attachment to the microscope body of the surgical microscope from FIG. 1, according to aspects of the present disclosure.

FIG. 7 shows a view from below of the attachment of the surgical microscope from FIG. 1 in the region of the interface, according to aspects of the present disclosure.

FIG. 8 shows a detail of the attachment from FIG. 7 in an enlarged illustration, according to aspects of the present disclosure.

FIGS. 9-12 show variants of attachments for the surgical microscope from FIG. 1, according to aspects of the present disclosure.

FIG. 13 shows the view in accordance with FIG. 3 in an alternative embodiment, according to aspects of the present disclosure.

FIG. 14 shows a further embodiment of a surgical microscope, according to aspects of the present disclosure.

### DETAILED DESCRIPTION

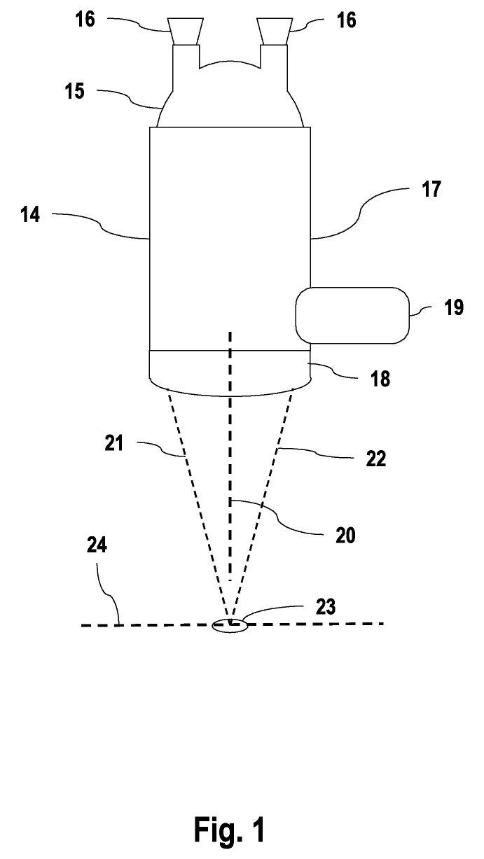

A surgical microscope 14 shown in FIG. 1 defines a stereoscopic observation beam path with a first beam path 21 for the left eye and a second beam path 22 for the right eye. The stereoscopic observation beam path 21, 22 extends from an object 23 in the form of a region of a patient’s body to be examined or treated, via a main objective 18, into a microscope body 17. An optical axis 20 of the surgical microscope 14 extends in the centre of the stereoscopic observation beam path 21, 22. The surgical microscope comprises a binocular head, referred to as a tube 15, on which two eyepieces 16 are formed. An observer directing their gaze through the two eyepieces 16 sees a magnified image from the object plane 24, in which the object 23 is arranged.

The surgical microscope 14 is equipped with an illumination system 19 which emits illumination light. The illumination light passes through the main objective 18 and illuminates the object plane 24. The image visible in the eyepieces 16 has sufficient brightness on account of the illumination light.

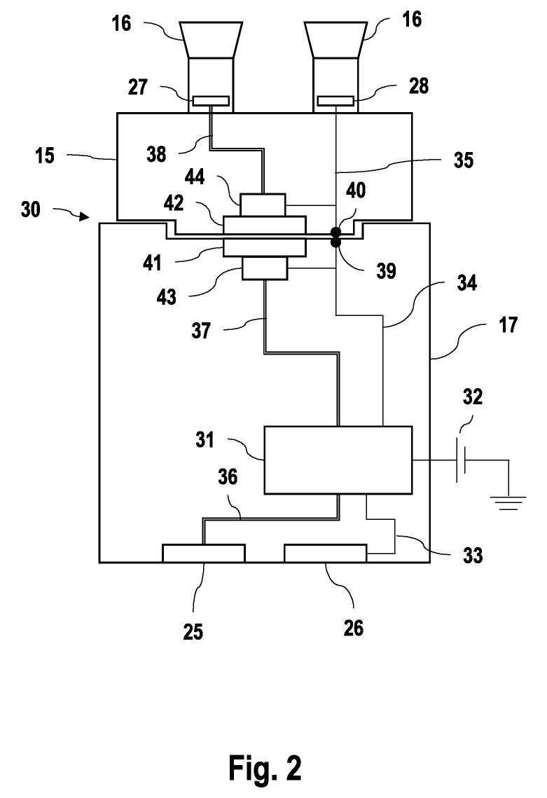

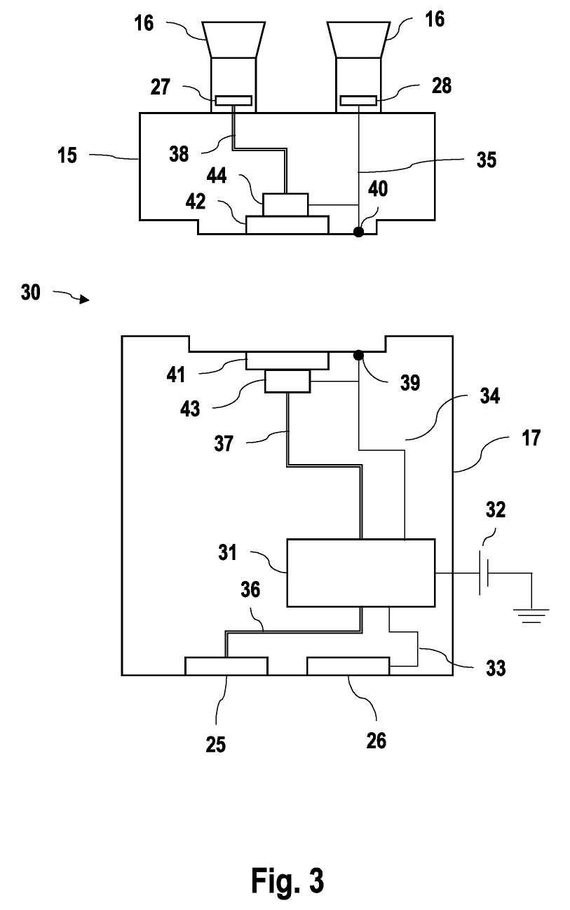

In accordance with FIGS. 2 and 3, the tube 15 is configured as an attachment 15 that is detachably connected to the microscope body 17 by way of an interface 30. FIG. 2 illustrates the tube 15 and the microscope body 17 in the connected state; FIG. 3 illustrates the tube 15 and the microscope body 17 in the separated state.

A first image sensor 25 for the left beam path 21 of the stereoscopic observation beam path and a second image sensor 26 for the right beam path 22 of the stereoscopic observation beam path are embodied in the microscope body 17. The optical elements of the surgical microscope 14 are configured such that the image sensors 25, 26 are located in an image plane of the observation beam path 21, 22. Images from the object plane 24 are captured by the image sensors 25, 26.

Each of the image sensors 25, 26 is connected to a control unit 31 of the surgical microscope 14 by way of a first signal line 36. Via a second signal line consisting of two sections 37, 38, the image data are transmitted by the control unit 31 to two displays 27, 28 that are arranged in the eyepieces 16 of the tube 15. In FIGS. 2 and 3, the signal lines 36, 37, 38 are only depicted for the left image sensor 25 and the left display 27. The right image sensor 26 and the right display 28 are connected to signal lines in a similar manner. The observer, who looks into the eyepieces 16, sees a stereoscopic image as a result of image data being displayed on two displays 27, 28.

The control unit 31 is connected to a voltage source 32. The image sensors 25, 26 are supplied with electrical energy by the control unit 31 via a first supply line 33. The displays 27, 28 are supplied with electrical energy by the control unit 31 via a second supply line, which comprises a first section 34 and a second section 35. The supply lines 33, 34, 35 are illustrated in FIGS. 2 and 3 only for the right image sensor 26 and the right display 28. The left image sensor 25 and the left display 27 are connected to supply lines in a similar manner.

The interface 30 between the tube 15 and the microscope body 17 is configured such that the first section 37 and the second section 38 of the signal line are separated from each other when the tube 15 is separated from the microscope body 17 and are connected to each other when the tube 15 is connected to the microscope body 17. Likewise, the first section 34 and the second section 35 of the supply line are separated from each other when the tube 15 is separated from the microscope body 17 and are connected to each other when the tube 15 is connected to the microscope body 17.

The first section 34 of the supply line terminates at a first connector contact 39 at the interface 30, and the second section 35 of the supply line terminates at a second connector contact 40 at an interface 30. The two sections 34, 35 of the supply line are connected and separated by way of the connector contacts 39, 40.

The first section 37 and the second section 38 of the signal lines are coupled by way of an optical interface. A first transducer 43, which is connected to the first section 37 of the signal lines, is arranged in the microscope body 17. In the first transducer 43, the electrical signal obtained via the first section 37 of the signal lines is converted into a light signal. The light signal is fed into a first glass block 41, which is a constituent part of the microscope body 17. The light signal propagates into a second glass block 42, which is a constituent part of the tube 15, via the first glass block 41 and a gap formed between the tube 15 and the microscope body 17. The light signal reaches a second transducer 44, in which the light signal is converted into an electrical signal again, via the second glass block 42. The electrical signal is conducted to the displays 27, 28 via the second section 38 of the supply line such that the transmitted image data can be displayed on the displays 27, 28.

The optical interface is configured so as to be contactless, and so a coupling of the optical interface is automatically established when the tube 15 is positioned correctly relative to the microscope body 17. Likewise, the optical interface is readily separated when the tube 15 is removed from the microscope body 17. Accordingly, the optical interface provides a data interface which enables a high transmission rate and at the same time can be connected or disconnected in an uncomplicated manner. The components of the optical interface are supplied with electricity via the supply line by virtue of the first transducer 43 being connected to the first section 34 of the supply line and by virtue of the second transducer 44 being connected to the second section 35 of the supply line.

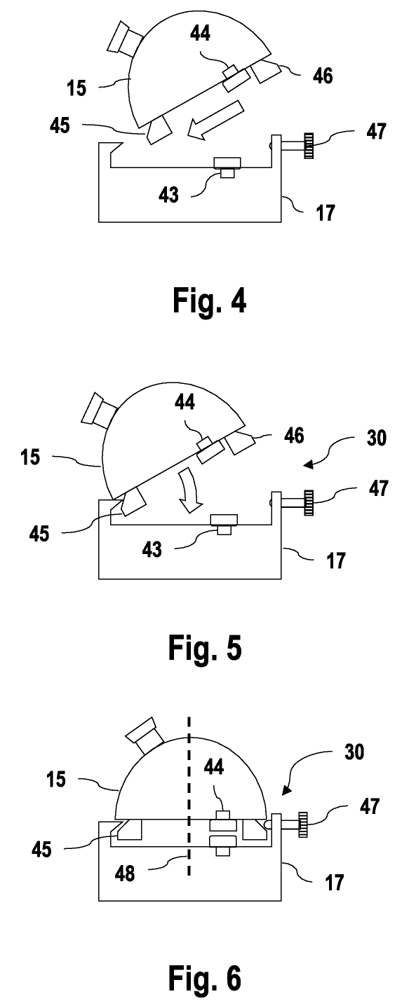

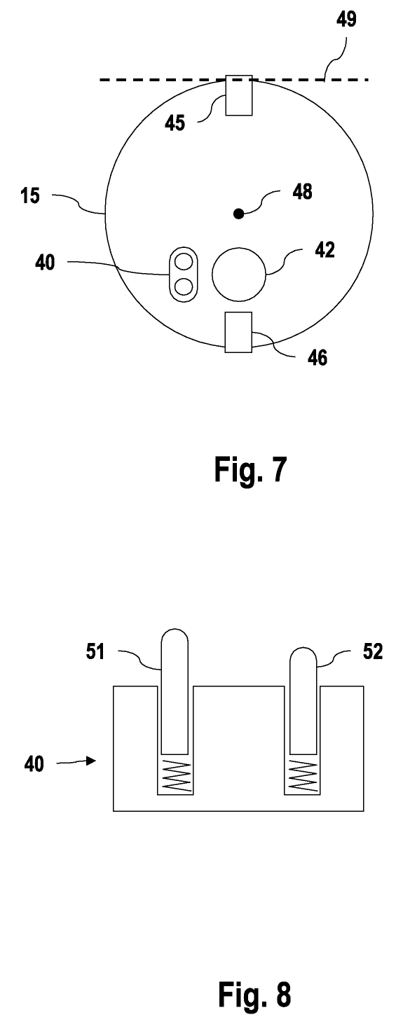

FIGS. 4-6 illustrate the movement sequence when connecting the tube 15 to the microscope body 17. In a first step, the tube 15, which is initially present separately, is brought to a receptacle of the microscope body 17 obliquely from above such that a first wedge element 45 of the tube 15 engages behind a counter element of the microscope body 17; see FIG. 5. The first wedge element 45 is a constituent part of a positioning structure within the meaning of the present disclosure. In a second step, the tube 15 is pivoted about a pivot axis 49 situated at the first wedge element 45 in order to bring the tube 15 into the correct alignment relative to the microscope body 17; see FIG. 6. The position of the pivot axis 49 is indicated in FIG. 7. A screw 47 attached to the microscope body 17 is tightened in order to clamp the tube 15 against the microscope body 17 by way of a second wedge surface 46. The tube 15 is thus fixed in its final position relative to the microscope body 17. The components 41-44 of the optical interface are arranged such that data transmission by way of a modulated light signal is possible.

The interface 30 between the tube 15 and the microscope body 17 has a central axis 48; see FIG. 6. The central axis 48 lies between the pivot axis 49 on the one side and the components 39, 40 of the electrical interface and the components 41-44 of the optical interface on the other side. What is achieved in this way is that the components 39, 40 of the electrical interface and the components 41-44 of the optical interface are brought closer together by way of a controlled movement.

The electrical interface 40 is designed as a two-pole interface with a first pin 51 as earth and a second pin 52 for the voltage signal. The two pins 51, 52 are designed differently such that the first pin 51 projects further forward than the second pin 52; see FIG. 8. In this way, the earth is connected first, as a result of which electrostatic charges can be dissipated before the circuit is closed by way of the second pin 52.

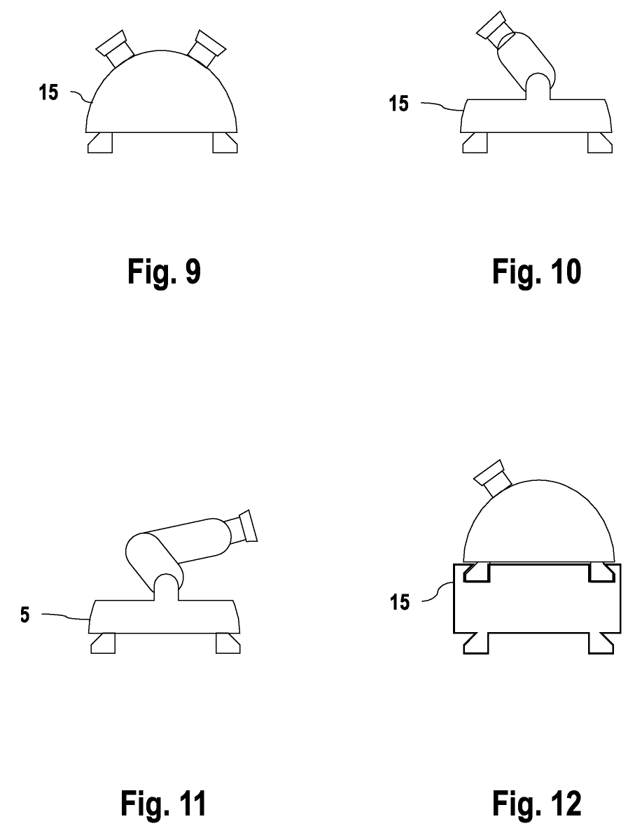

The interface 30 opens up the possibility of changing between different types of attachment 15 on the microscope body 17 as required. FIGS. 9-12 show various types of attachments 15 which can be connected to the microscope body 17 by way of the interface 30. FIG. 9 shows an attachment in the form of a tube 15 with two pairs of eyepieces such that two users can see the microscope image at the same time. In the case of the tube 15 in FIG. 10, the eyepieces are pivotably mounted on a base of the tube 15. In FIG. 11, the position and orientation of the eyepieces can be variably set by way of two pivot joints. In FIG. 12, the attachment comprises an intermediate piece which can optionally be inserted between the tube and the microscope body 17 or be omitted. Use of the intermediate piece leads to two interfaces according to the present disclosure being present between the tube and the microscope body 17.

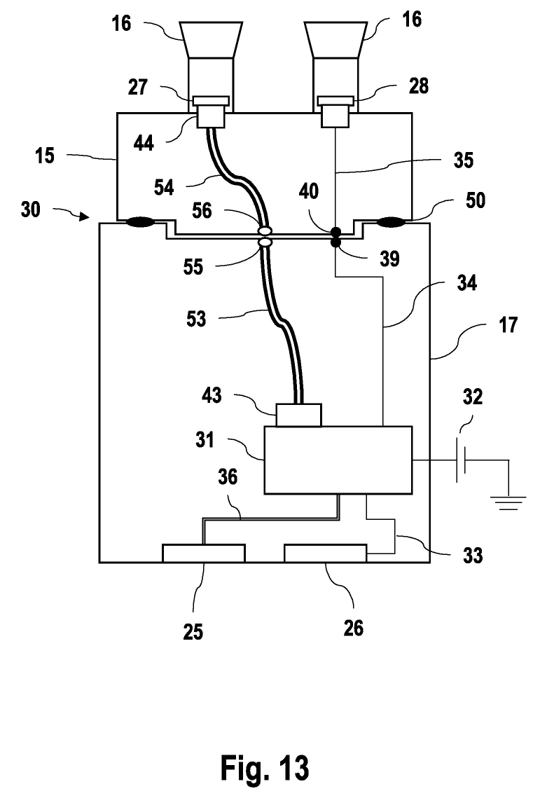

FIG. 13 illustrates an alternative embodiment of a surgical microscope according to the present disclosure, in which the first transducer 43 is arranged adjacent to the control unit 31 of the surgical microscope and in which the second transducer 44 is arranged adjacent to the displays 27, 28. The light signal is transmitted between the first transducer 43 and the second transducer 44 via an optical conductor path which is composed of a first light guide 53 in the microscope body 17 and a second light guide 54 in the tube 15. The exit end of the first light guide 53 arranged at the interface 30 is provided with a first collimating optical unit, via which the light signal emerges in the direction of the second light guide 54. The entrance end of the second light guide 54, which is provided with a second collimating optical unit 56, is arranged opposite the exit end of the first light guide 53. The light signal is captured by the second collimating optical unit 56 and introduced into the second light guide 54. The path length between the first transducer 43 and the second transducer 44 is less than 1 m, for which reason the light guides 53, 54 can be formed as cost-effective polymer light guides. The interface 30 is provided with a continuous peripheral sealing ring 50, by means of which an ingress of liquids into the central region of the interface 30 is prevented. The optical interface and the electrical interface are located within the sealing ring 50 and hence in a region which is protected from liquids.

In this exemplary embodiment, the electrical interface 39, 40 is provided with four pins. Two of the pins serve to supply the electronic components in the tube 15 with electrical energy. Control signals are transmitted via the two other pins. For example, the content of the control signals can be an identification code about the type of attached attachment 15. This may open up the possibility of identifying the attachment 15 without the control unit 31 being in a fully active operating mode.

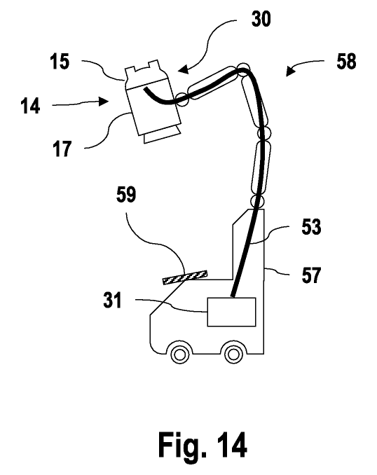

In the alternative embodiment in accordance with FIG. 14, the surgical microscope 14 is suspended from a stand comprising a stand base 57 and a stand arm 58. The stand has an operating unit 59, by means of which actuators of the stand arm 58 can be actuated in order to change the position and the orientation of the surgical microscope 14. The control unit 31 of the surgical microscope is arranged in the stand base 57. The image data are transmitted in the direction of the attachment 15 via a first light guide 53, which extends from the control unit 31 along the stand arm 58 to the interface 30 between the microscope body 17 and the attachment 15. The first light guide 53 has a length of several metres. The first light guide 53 is made of glass fibres in order nevertheless to enable a reliable, low-loss transmission of the light signal. The second light guide 54, which extends between the interface 30 and the eyepiece 16, is shorter and can consist of a polymer or glass fibre.

The foregoing description of the embodiments, including illustrated embodiments, has been presented only for the purpose of illustration and description and is not intended to be exhaustive or limiting to the precise forms disclosed. Numerous modifications, adaptations, and uses thereof will be apparent to those skilled in the art. Numerous changes to the disclosed embodiments can be made in accordance with the disclosure herein, without departing from the spirit or scope of the disclosure. Thus, the breadth and scope of the present disclosure should not be limited by any of the above described embodiments.

Although certain aspects and features of the present disclosure have been illustrated and described with respect to one or more implementations, equivalent alterations and modifications will occur or be known to others skilled in the art upon the reading and understanding of this specification and the annexed drawings. In addition, while a particular feature may have been disclosed with respect to only one of several implementations, such feature may be combined with one or more other features of the other implementations as may be desired and advantageous for any given or particular application.

The terminology used herein is for the purpose of describing particular embodiments only, and is not intended to be limiting of the disclosure. As used herein, the singular forms “a,” “an,” and “the” are intended to include the plural forms as well, unless the context clearly indicates otherwise. Furthermore, to the extent that the terms “including,” “includes,” “having,” “has,” “with,” or variants thereof, are used in either the detailed description and/or the claims, such terms are intended to be inclusive in a manner similar to the term “comprising.”

One or more elements or aspects or steps, or any portion(s) thereof, from one or more of any of the claims below can be combined with one or more elements or aspects or steps, or any portion(s) thereof, from one or more of any of the other claims below or combinations thereof, to form one or more additional implementations and/or claims of the present disclosure.

What is claimed is:

1. A surgical microscope comprising:
a microscope body; and
an attachment;
wherein the attachment is detachably attached to the microscope body by way of an interface,
wherein the microscope body is provided with an optical arrangement and an image sensor,
wherein the optical arrangement guides an observation beam path that extends between an object plane and the image sensor,
wherein the attachment comprises a display for displaying image data acquired by the image sensor,
wherein the interface comprises a separable electrical channel and a separable optical data channel, and
wherein the image data are transmitted along the optical data channel via the interface.

2. The surgical microscope according to claim 1, further comprising a first transducer for converting an electrical signal of the image data into an optical signal of the image data.

3. The surgical microscope according to claim 1, further comprising a second transducer for converting an optical signal of the image data into an electrical signal of the image data.

4. The surgical microscope according to claim 3, wherein the second transducer is an element of the attachment.

5. The surgical microscope according to claim 1, wherein a separation point corresponding to the interface is formed in the optical data channel.

6. The surgical microscope according to claim 1, wherein the optical data channel comprises a light guide, the light guide being arranged on a section of the optical data channel.

7. The surgical microscope according to claim 6, wherein the light guide is made of a polymer, a glass fibre, or a combination thereof.

8. The surgical microscope according to any of Claims claim 1, wherein the electrical channel transmits the electrical energy required for the operation of the display.

9. The surgical microscope according to claim 1, wherein the electrical channel transmits control information between the attachment and the microscope body.

10. The surgical microscope according to claim 1, further comprising a positioning structure which, in a second engagement position, allows the attachment to be affixed to the microscope body by means of a locking mechanism, and which, in a first engagement position, prevents an engagement of the locking mechanism.

11. The surgical microscope according to claim 10, wherein transition between the first engagement position and the second engagement position is effected by a pivoting movement about a pivot axis.

12. The surgical microscope according to claim 11, wherein, within the interface, the distance between the optical data channel and the pivot axis is greater than the distance between a central axis of the interface and the pivot axis.

13. The surgical microscope according to claim 12, wherein, within the interface, the distance between the electrical channel and the pivot axis is greater than the distance between a central axis of the interface and the pivot axis.

14. A method for operating a surgical microscope,
wherein the surgical microscope comprises a microscope body and an attachment,
wherein the attachment is detachably attached to the microscope body by way of an interface,
wherein image data representing images from an object plane of the surgical microscope are acquired by an image sensor,
wherein the image data are transmitted via the interface to a display formed in the attachment,
wherein image data are transmitted via an optical data channel formed in the interface, and
wherein the display is supplied with electrical energy via an electrical channel formed in the interface.
